(19) United States
(12) Patent Application Publication
(10) Pub. No.: US 2024/0261244 A1
(43) Pub. Date: Aug. 8, 2024

(54) METHODS FOR TREATING VIRAL INFECTIONS

(71) Applicant: Daarsh Innovations, Inc., Ridgewood, NJ (US)

(72) Inventors: Dinesh AGGARWAL, Ridgewood, NJ (US); Nitin BHATIA, Ridgewood, NJ (US)

(21) Appl. No.: 18/430,778

(22) Filed: Feb. 2, 2024

Related U.S. Application Data
(60) Provisional application No. 63/442,822, filed on Feb. 2, 2023.

(51) Int. Cl. A61K 31/198; A61K 31/047; A61P 31/14

(52) U.S. Cl. CPC A61K 31/198; A61K 31/047; A61P 31/14

(57) ABSTRACT

A method for reducing a viral titer in a subject by administering a composition comprising a sodium salt of Ethylenediaminetetraacetic acid (EDTA), D-mannitol and Ethanol. Also, a method for treating a subject having a viral infection by administering the composition comprising a sodium salt of Ethylenediaminetetraacetic acid (EDTA), D-mannitol and Ethanol. Further, a composition comprising a sodium salt of Ethylenediaminetetraacetic acid (EDTA), D-mannitol and Ethanol for use in treating a viral infection.

### CROSS-REFERENCE TO RELATED APPLICATIONS

The present application claims the benefit of priority from U.S. Provisional Application No. 63/442,822, filed Feb. 2, 2023, the contents of which are hereby incorporated by reference in its entirety.

### FIELD OF THE DISCLOSURE

The present invention relates to a use of a composition comprising a sodium salt of ethylenediaminetetraacetic acid (EDTA), D-mannitol and ethanol for treating a viral infection. In particular, the present invention relates to a use of the composition comprising a sodium salt of ethylenediaminetetraacetic acid (EDTA), D-mannitol and ethanol for treating respiratory viral infections, for example, caused by coronavirus, influenza, respiratory syncytial virus (RSV), rhinovirus.

### BACKGROUND OF THE DISCLOSURE

Viral diseases causing pandemics (e.g., AIDS, 2009 H1N1 pandemic flu, COVID-19) are of major concern for human health. Viruses are continuously evolving, which has a direct impact on their pathogenic potential. The implications of viral infections can be extraordinarily diverse ranging from mildly symptomatic infections to death.

Viruses exhibit an extensive diversity in structure and lifecycle. They primarily consist of a small segment of nucleic acid encased in a protein shell (capsid). They can be broadly classified into enveloped (having a lipid-bilayer membrane surrounding the capsid) and non-enveloped (lacking a lipid-bilayer membrane, i.e., the capsid is not surrounded by a lipid-bilayer). These structural differences result in different mechanisms of cell entry and different pathways of assembly and maturation.

Enveloped viruses enter by membrane fusion, either from an internal compartment, or at the cell surface. Non-enveloped viruses require some form of membrane “perforation”. Much of the specificity of a virus for a certain type of cell lies in its ability to attach to the surface of that specific cell.

Durable contact is important for the virus to infect the host cell, and the ability of the virus and the cell surface to interact is a property of both the virus and the host cell. The fusion of viral and host-cell membranes allows the intact viral particle, or, in certain cases, only its infectious nucleic acid to enter the cell.

Coronavirus causes variety of illnesses ranging from common cold to Covid-19. It is an enveloped virus. Human rhinovirus cause many common viral infections. It is a type of non-enveloped virus. It is believed that both corona and rhinovirus enter into humans via the upper respiratory tract particularly the nose and the mouth.

Numerous viral infections are spread by direct contact between an infected person and healthy individual by hand-to-hand, hand-to-mouth, aerosol droplet transfer, or by indirect contact when an uninfected individual comes in contact with a virus-contaminated solid object.

Therefore, in order to control a viral infection, it is important to rapidly eliminate a virus.

One of the common viral infections include sinusitis/rhinosinusitis. Sinusitis affects about 1 in 8 adults in the United States, resulting in over 30 million annual diagnoses. The direct cost of managing acute and chronic sinusitis exceeds $11 billion per year, with additional expense from lost productivity and impaired quality of life. More than 1 in 5 antibiotics prescribed in adults are for sinusitis, making it the fifth most common diagnosis responsible for antibiotic therapy. The broad category of rhinosinusitis includes Acute Rhinosinusitis (ARS) and Chronic Rhinosinusitis (CRS). Most ARS begins when a viral upper respiratory infection (URI) extends into the paranasal sinuses, which may be followed by bacterial infection. About 20 million cases of presumed bacterial ARS (ABRS) occur annually in the United States, rendering it one of the most common conditions encountered by clinicians. National ambulatory care data from 2006 to 2010 revealed that rhinosinusitis accounted for more outpatient antibiotic prescriptions than any other diagnosis. Despite guidelines that encourage judicious antibiotic use for ARS, they are prescribed in about 82% of visits. ARS and CRS combined accounted for more primary ambulatory care visits with antibiotic prescriptions than any other diagnosis or commonly grouped diagnoses. ARS has significant economic implications. The cost of antibiotic treatment failure, including additional prescriptions, outpatient visits, tests, and procedures, contributes to a substantial total ARS-related health care expenditure of more than $3 billion per year in the United States. CRS also has significant socioeconomic implications. In 2001, there were 18.3 million office visits for CRS, most of which resulted in prescription medications. Patients with CRS visit primary care clinicians twice as often as those without the disorder and have 5 times as many prescriptions filled. A survey in 2007 found that approximately $8.3 billion is spent annually on CRS, primarily on prescription drugs and office-based care.

Despite the vast healthcare burden of sinus infections, there is no FDA approved intranasal antimicrobial therapy. Current FDA approved treatment options include intranasal and oral steroids, intranasal and oral antihistamines, and oral antibiotics, but no intranasal antibiotics or antimicrobials. The vast majority of sinusitis is caused by viral pathogens followed by bacterial pathogens, and fungal sinus infections are commonly seen in immunocompromised patients. There is an unmet need for treating bacterial, viral, and fungal infections in the nasal cavity by providing effective nasal formulations, thereby providing an easy-to-use treatment option with minimal systemic side effects. Also, there is an unmet need for treating bacterial, viral, and fungal infections of the skin by providing effective topical formulations that can rapidly eliminate these pathogens. The present invention attempts to address these needs.

### SUMMARY OF THE DISCLOSURE

The present disclosure provides a method for reducing a viral titer in a subject comprising administering to the subject a composition comprising a sodium salt of ethylenediaminetetraacetic acid (EDTA), D-mannitol and ethanol.

The present disclosure provides a method for treating a subject having a viral infection comprising administering to the subject a composition comprising a sodium salt of ethylenediaminetetraacetic acid (EDTA), D-mannitol and ethanol.

The present disclosure further provides a composition for use in treating a viral infection, wherein the composition comprises a sodium salt of Ethylenediaminetetraacetic acid (EDTA), D-mannitol and ethanol.

The methods and the composition for use of the present disclosure show an antiviral activity against enveloped and non-enveloped viruses, wherein the viruses are coronavirus (e.g., SARS-CoV-2), influenza virus, respiratory syncytial virus (RSV), rhinovirus, or variants thereof.

### BRIEF DESCRIPTION OF THE DRAWINGS

FIG. 1: The FIGURE provides the graphical representation of the antiviral activity using the present formulation in comparison to 70% v/v ethanol, 5 wt % EDTA, 25 v/v % ethanol, and control.

### DETAILED DESCRIPTION OF THE DISCLOSURE

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The use of the expression “at least” or “at least one” suggests the use of one or more elements or ingredients or quantities, as the use may be in the embodiment of the disclosure to achieve one or more of the desired objects or results. Throughout this specification, the word “comprise”, or variations such as “comprises” or “comprising” or “containing” or “has” or “having”, or “including but not limited to” wherever used, will be understood to imply the inclusion of a stated element, integer or step, or group of elements, integers or steps, but not the exclusion of any other element, integer or step, or group of elements, integers or steps.

Reference throughout this specification to “one embodiment”, “an embodiment”, or “some embodiments” means that a particular feature, structure or characteristic described in connection with the embodiment may be included in at least one embodiment of the present disclosure. Thus, the appearances of the phrases “in one embodiment”, “in an embodiment”, or “in some embodiments” in various places throughout this specification may not necessarily all refer to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

The term “subject” or “patient” as used herein refers to any mammal including, without limitation, humans and other primates (e.g., chimpanzees and other apes and monkey species), farm animals (e.g., cattle, sheep, pigs, goats and horses), domestic mammals (e.g., dogs and cats), and laboratory animals (e.g., rodents such as mice, rats, and guinea pigs). In some embodiments, the patient is a mammal. In some embodiments, the patient is a human.

The term “anti-infective” as used herein refers to substances/agents, used either alone or in combination with other materials such as carriers, solvents, or the like, which lead to reduction, inhibition, or elimination of infectious microbes from a subject.

The term “antiviral” as used herein refers to a composition or agent that kills or otherwise inhibits the growth of viruses.

The term “virucidal activity” is used to refer to an activity that reduces or inhibits virus plaque forming units or virus particles. Contamination of surfaces, e.g., skin, hands, nasal passage, oral cavity, other inanimate surfaces pose serious and substantial health risks and virucidal disinfection is a significant priority.

The term “about” as used herein encompasses variations of +/−10% and more preferably +/−5%, as such variations are appropriate for practicing the present invention.

In some embodiments, the present disclosure provides a method for reducing a viral titer in a subject comprising administering to the subject a composition comprising a sodium salt of ethylenediaminetetraacetic acid (EDTA), D-mannitol and ethanol.

In some embodiments, the present disclosure provides a method for treating a subject having a viral infection comprising administering to the subject a composition comprising a sodium salt of ethylenediaminetetraacetic acid (EDTA), D-mannitol and ethanol. In some embodiments, the viral infection is a nasal infection or a skin infection. In some embodiments, the viral infection is a nasal infection. In some embodiments, the viral infection is a skin infection.

In some embodiments, the administration of the composition reduces the viral titer of or treats a viral infection caused by enveloped and non-enveloped viruses. In some embodiments, the composition reduces the viral titer of or treats a viral infection caused by enveloped or non-enveloped viruses selected from the group consisting of coronavirus, influenza virus, respiratory syncytial virus (RSV), rhinovirus, herpes simplex virus (HSV), human papilloma virus (HPV), varicella zoster virus (VZV), and variants thereof. In some embodiments, the composition reduces the viral titer of or treats a viral infection caused by a virus selected from the group consisting of SARS-CoV-2, influenza virus, respiratory syncytial virus (RSV), rhinovirus, and variants thereof.

In some embodiments, the administration of the composition reduces the viral titer of or treats a viral infection caused by a coronavirus including SARS-CoV-2 or variants thereof. In some embodiments, the administration of the composition reduces the viral titer of or treats a viral infection caused by a SARS-CoV-2 or variants thereof.

In some embodiments, the administration of the composition reduces the viral titer of or treats a viral infection caused by an influenza virus. In some embodiments, the administration of the composition reduces the viral titer of or treats a viral infection caused by influenza A virus including influenza A (H1N1), influenza A (H3N2) or influenza B virus.

In some embodiments, the administration of the composition reduces the viral titer of or treats the viral infection causing skin infections. In some embodiments, the administration of the composition reduces the viral titer of or treats a viral infection caused by herpes simplex virus (HSV), human papilloma virus (HPV), varicella zoster virus (VZV), or variants thereof.

In some embodiments, the administration of the composition reduces the viral titer in the subject by at least about 60%, 65%, 70%, 75% 80%, 85%, 90% 95%, 99%, 99.99%, or 100% compared to levels of viral titer prior to administration of the composition. In some embodiments, the administration of the composition reduces the viral titer by 60-99.99%, 60-99%, 60-95%, 60-90%, 60-85%, 60-80%, 60-75%, 60-70%, 60-65%, 65-99.99%, 65-99%, 65-95%, 65-90%, 65-85%, 65-80%, 65-75%, 65-70%, 70-99.99%, 70-99%, 70-95%, 70-90%, 70-85%, 70-80%, 70-75%, 75-99.99%, 75-99%, 75-95%, 75-90%, 75-85%, 75-80%, 80-99.99%, 80-99%, 80-95%, 80-90%, 80-85%, 85-99.99%, 85-99%, 85-95%, 85-90%, 90-99.99%, 90-99%, 90-95%, 95-99%, or 99-99.99%, including values and ranges thereof, compared to levels of viral titer prior to administration of the composition. In some embodiments, the administration of the composition reduces the viral titer by about 80%. In some embodiments, the administration of the composition reduces the viral titer by about 95%.

In some embodiments, the administration of the composition reduces the viral titer in the subject in at least about 0.5 minute, 0.75 minute, 1 minute, 2 minutes, 3 minutes, 5 minutes, or 10 minutes after the administration of the composition. In some embodiments, the administration of the composition reduces the viral titer in about 0.5-0.75 minute, 0.5-1 minute, 0.5-2 minutes, 0.5-3 minutes, 0.5-5 minutes, 0.5-10 minutes, 0.75-1 minute, 0.75-2 minutes, 0.75-3 minutes, 0.75-5 minutes, 0.75-10 minutes, 1-2 minutes, 1-3 minutes, 1-5 minutes, 1-10 minutes, 2-3 minutes, 2-5 minutes, 2-10 minutes, 3-5 minutes, 3-10 minutes, or 5-10 minutes, including values and ranges thereof, after the administration of the composition. In some embodiments, the administration of the composition reduces the viral titer in the subject by at least 60% in about 0.5 minute compared to levels of viral titer prior to administration of the composition. In some embodiments, the administration of the composition reduces the viral titer in the subject by about 80% in about 5 minutes compared to levels of viral titer prior to administration of the composition. In some embodiments, the administration of the composition reduces the viral titer in the subject by about 100% in about 3 minutes compared to levels of viral titer prior to administration of the composition. In some embodiments, the administration of the composition reduces the viral titer in the subject by about 100% in about 5 minutes compared to levels of viral titer prior to administration of the composition. In some embodiments, the administration of the composition reduces the viral titer in the subject by about 100% in about 10 minutes compared to levels of viral titer prior to administration of the composition.

In some embodiments, the administration of the composition reduces the viral titer in the subject by at least about 60%, 65%, 70%, 75% 80%, 85%, 90% 95%, 99%, 99.99%, or 100% in about at least 0.5 minute, 0.75 minute, 1 minute, 2 minutes, 3 minutes, 5 minutes, or 10 minutes after the administration of the composition compared to levels of viral titer prior to administration of the composition. In some embodiments, the administration of the composition reduces the viral titer in the subject by 60-99.99%, 60-99%, 60-95%, 60-90%, 60-85%, 60-80%, 60-75%, 60-70%, 60-65%, 65-99.99%, 65-99%, 65-95%, 65-90%, 65-85%, 65-80%, 65-75%, 65-70%, 70-99.99%, 70-99%, 70-95%, 70-90%, 70-85%, 70-80%, 70-75%, 75-99.99%, 75-99%, 75-95%, 75-90%, 75-85%, 75-80%, 80-99.99%, 80-99%, 80-95%, 80-90%, 80-85%, 85-99.99%, 85-99%, 85-95%, 85-90%, 90-99.99%, 90-99%, 90-95%, 95-99%, or 95-99.99% in about 0.5-0.75 minute, 0.5-1 minute, 0.5-2 minutes, 0.5-3 minutes, 0.5-5 minutes, 0.5-10 minutes, 0.75-1 minute, 0.75-2 minutes, 0.75-3 minutes, 0.75-5 minutes, 0.75-10 minutes, 1-2 minutes, 1-3 minutes, 1-5 minutes, 1-10 minutes, 2-3 minutes, 2-5 minutes, 2-10 minutes, 3-5 minutes, 3-10 minutes, or 5-10 minutes, including values and ranges thereof, after the administration of the composition.

In some embodiments, the administration of the composition reduces the viral titer in the subject by at least 1 log₁₀, 2 log₁₀, 3 log₁₀, 4 log₁₀, 5 log₁₀, 6 log₁₀, 8 log₁₀, or 10 log₁₀ as measured according to a Plaque Assay testing method. In some embodiments, the administration of the composition reduces the viral titer in the subject by 1 log₁₀−2 log₁₀, 1 log₁₀−3 log₁₀, 1 log₁₀−4 log₁₀, 1 log₁₀−5 log₁₀, 1 log₁₀−6 log₁₀, 1 log₁₀−8 log₁₀, 1 log₁₀−10 log₁₀, 2 log₁₀−3 log₁₀, 2 log₁₀−4 log₁₀, 2 log₁₀−5 log₁₀, 2 log₁₀−6 log₁₀, 2 log₁₀−8 log₁₀, 2 log₁₀−10 log₁₀, 3 log₁₀−4 log₁₀, 3 log₁₀−5 log₁₀, 3 log₁₀−6 log₁₀, 3 log₁₀−8 log₁₀, 3 log₁₀−10 log₁₀, 4 log₁₀−5 log₁₀, 4 log₁₀−6 log₁₀, 4 log₁₀−8 log₁₀, 4 log₁₀−10 log₁₀, 5 log₁₀−6 log₁₀, 5 log₁₀−8 log₁₀, 5 log₁₀−10 log₁₀, 6 log₁₀−8 log₁₀, 6 log₁₀−10 log₁₀, or 8 log₁₀−10 log₁₀ including values and ranges thereof, after the administration of the composition.

In some embodiments the Plaque Assay testing method comprises of following steps:
1. Plating Vero E6 cells onto 96-well plates and incubating overnight at 37° C.
2. Exposing Vero E6 cells to virus-test solution mixture, virus-control mixture (positive control) and virus only (negative control).
3. Restricting the virus growth to infected cells by addition of growth media and Carboxy methylcellulose mixture and incubation for 3 days.
4. Fixing the cells by addition of formaldehyde and incubation for 30 min.
5. Staining via crystal violet for plaques visualization and PFU/ml determination.

In some embodiments, the methods of the present disclosure comprise administering the compositions described herein intranasally, enterally, sublingually, intramuscularly, intravenously, intrathecally, topically, as irrigation, subcutaneously, via inhalation, or mucosally.

In some embodiments, the methods of the present disclosure comprise administering the compositions described herein intranasally. Accordingly, in some embodiments, the compositions described herein are formulated for intranasal administration such as in the form of a nasal spray, nasal mist, nasal drops, nasal gels, nasal irrigation solutions, nasal suspensions and emulsion, and the like.

In some embodiments, the composition is administered at least once a day, at least twice a day, at least three times a day or at least four times a day. The frequency of administration of the composition can vary based on a variety of factors, such as, age, weight, sex, a medical condition of the patient, etc.

As described above, the present methods comprise administration of the composition comprising a sodium salt of ethylenediaminetetraacetic acid (EDTA), D-mannitol and ethanol. In some embodiments, the sodium salt of EDTA is selected from the group consisting of di-sodium salt of Ethylenediaminetetraacetic acid (EDTA), tri-sodium salt of Ethylenediaminetetraacetic acid (EDTA), tetra-sodium salt of Ethylenediaminetetraacetic acid (EDTA), and a combination thereof. In some embodiments, the sodium salt of EDTA is di-sodium salt of Ethylenediaminetetraacetic acid (EDTA).

In some embodiments, the sodium salt of EDTA is present in the composition at a concentration ranging from about 4% to about 8% (w/v). In some embodiments, the sodium salt of EDTA is present in the composition at a concentration ranging from 4-5% (w/v), 4-6% (w/v), 4-7% (w/v), 4-8% (w/v), 5-6% (w/v), 5-7% (w/v), 5-8% (w/v), 6-7% (w/v), 6-8% (w/v), or 7-8% (w/v), including values and ranges thereof. In some embodiments, the sodium salt of EDTA is present in the composition at a concentration of about 5% (w/v). In some embodiments, the sodium salt of EDTA is present in the composition at a concentration of 5% (w/v).

In some embodiments, D-mannitol is present in the composition at a concentration ranging from about 0.5% to about 10% (w/v). In some embodiments, D-mannitol is present in the composition at a concentration ranging from 0.5-1% (w/v), 0.5-1.5% (w/v), 0.5-2% (w/v), 0.5-2.5% (w/v), 0.5-3% (w/v), 0.5-3.5% (w/v), 0.5-4% (w/v), 0.5-4.5% (w/v), 0.5-5% (w/v), 0.5-5.5% (w/v), 0.5-6% (w/v), 0.5-6.5% (w/v), 0.5-7% (w/v), 0.5-7.5% (w/v), 0.5-8% (w/v), 0.5-8.5% (w/v), 0.5-9% (w/v), 0.5-9.5% (w/v), 0.5-10% (w/v), 1-1.5% (w/v), 1-2% (w/v), 1-3% (w/v), 1-4% (w/v), 1-5% (w/v), 1-6% (w/v), 1-7% (w/v), 1-8% (w/v), 1-9% (w/v), 1-10% (w/v), 1.5-2% (w/v), 1.5-3% (w/v), 1.5-4% (w/v), 1.5-5% (w/v), 1.5-6% (w/v), 1.5-7% (w/v), 1.5-8% (w/v), 1.5-9% (w/v), 1.5-10% (w/v), 2-3% (w/v), 2-4% (w/v), 2-5% (w/v), 2-6% (w/v), 2-7% (w/v), 2-8% (w/v), 2-9% (w/v), 2-10% (w/v), 3-4% (w/v), 3-5% (w/v), 3-6% (w/v), 3-7% (w/v), 3-8% (w/v), 3-9% (w/v), 3-10% (w/v), 4-5% (w/v), 4-6% (w/v), 4-7% (w/v), 4-8% (w/v), 4-9% (w/v), 4-10% (w/v), 5-6% (w/v), 5-7% (w/v), 5-8% (w/v), 5-9% (w/v), 5-10% (w/v), 6-7% (w/v), 6-8% (w/v), 6-9% (w/v), 6-10% (w/v), 7-8% (w/v), 7-9% (w/v), 7-10% (w/v), 8-9% (w/v), 8-10% (w/v), or 9-10% (w/v), including values and ranges thereof. In some embodiments, D-mannitol is present in the composition at a concentration of about 0.75% (w/v). some embodiments, D-mannitol is present in the composition at a concentration of 0.75% (w/v).

In some embodiments, ethanol is present in the composition at a concentration ranging from about 12% to about 25% (v/v). In some embodiments, ethanol is present in the composition at a concentration ranging from 12-12.5% (v/v), 12-13% (v/v), 12-14% (v/v), 12-15% (v/v), 12-16% (v/v), 12-17% (v/v), 12-18% (v/v), 12-19% (v/v), 12-20% (v/v), 12-21% (v/v), 12-22% (v/v), 12-23% (v/v), 12-24% (v/v), 12-25% (v/v), 14-15% (v/v), 14-16% (v/v), 14-17% (v/v), 14-18% (v/v), 14-19% (v/v), 14-20% (v/v), 14-21% (v/v), 14-22% (v/v), 14-23% (v/v), 14-24% (v/v), 14-25% (v/v), 16-17% (v/v), 16-18% (v/v), 16-19% (v/v), 16-20% (v/v), 16-21% (v/v), 16-22% (v/v), 16-23% (v/v), 16-24% (v/v), 16-25% (v/v), 18-19% (v/v), 18-20% (v/v), 18-21% (v/v), 18-22% (v/v), 18-23% (v/v), 18-24% (v/v), 18-25% (v/v), 20-21% (v/v), 20-22% (v/v), 20-23% (v/v), 20-24% (v/v), 20-25% (v/v), 22-23% (v/v), 22-24% (v/v), 22-25% (v/v), or 24-25% (v/v), including values and ranges thereof. In some embodiments, ethanol is present in the composition at a concentration of about 25% (v/v). In some embodiments, ethanol is present in the composition at a concentration of 25% (v/v).

In some embodiments, the composition has a pH ranging from about 4 to 12. In some embodiments, the composition has a pH ranging from about 4-5, 4-6, 4-7, 4-8, 4-9, 4-10, 4-11, 4-12, 5-6, 5-7, 5-8, 5-9, 5-10, 5-11, 5-12, 6-7, 6-8, 6-9, 6-10, 6-11, 6-12, 7-8, 7-9, 7-10, 7-11, 7-12, 8-9, 8-10, 8-11, 8-12, 9-10, 9-11, 9-12, 10-11, 10-12, or 11-12, including values and ranges thereof. In some embodiments, the composition has a pH of about 7.5+/−0.5. In some embodiments, the composition has a pH of 7.5+/−0.5.

In some embodiments, the composition comprises the sodium salt of EDTA at a concentration ranging from about 4% to about 8% (w/v), D-mannitol at a concentration ranging from about 0.5% to about 10% (w/v), and ethanol at a concentration ranging from about 12.5% to about 25% (v/v), including values and ranges thereof for each of these components as described in the aforementioned embodiments.

In some embodiments, the composition comprises the sodium salt of EDTA at a concentration of about 5% (w/v), D-mannitol at a concentration of about 0.75% (w/v), and ethanol at a concentration of about 25% (v/v).

In some embodiments, the composition comprises pharmaceutically acceptable excipients. In some embodiments, the pharmaceutically acceptable excipients are selected from, but not limited to, distilled water, glycerin or phosphate buffer saline, or a combination thereof.

In some embodiments, the composition may be used for topical applications in the form of creams, emollients, or other skin care compositions by adding pharmaceutically acceptable agents such as aloe-vera, and the like.

In some embodiments, the present disclosure provides a composition for use in treating a viral infection, wherein the composition comprises a sodium salt of Ethylenediaminetetraacetic acid (EDTA), D-mannitol and ethanol. The values and ranges for the amounts of the sodium salt of Ethylenediaminetetraacetic acid (EDTA), D-mannitol and ethanol present in the composition for use in treating a viral infection are as described above. The pH of the composition for use in treating a viral infection is as described above.

The frequency of administration and the routes of administration of the composition comprising a sodium salt of Ethylenediaminetetraacetic acid (EDTA), D-mannitol and ethanol for use in treating a viral infection are as described above.

In some embodiments, the composition for use in treating a viral infection reduces the viral titer of or shows an antiviral activity against enveloped and non-enveloped viruses.

In some embodiments, provided herein is a composition for use in treating a viral infection, wherein the composition comprises a sodium salt of Ethylenediaminetetraacetic acid (EDTA), D-mannitol and ethanol, and wherein the viral infection is caused by a virus selected from the group consisting of coronavirus, influenza, respiratory syncytial virus (RSV), rhinovirus, and variants thereof. In some embodiments, provided herein is a composition for use in treating a viral infection caused by a coronavirus including SARS-CoV-2 or variants thereof, and wherein the composition comprises a sodium salt of Ethylenediaminetetraacetic acid (EDTA), D-mannitol and ethanol. In some embodiments, provided herein is a composition for use in treating a viral infection caused by a SARS-CoV-2 or variants thereof, and wherein the composition comprises a sodium salt of Ethylenediaminetetraacetic acid (EDTA), D-mannitol and ethanol.

In some embodiments, provided herein is a composition for use in treating a viral infection, wherein the viral infection is a nasal infection or a skin infection, and wherein the composition comprises a sodium salt of Ethylenediaminetetraacetic acid (EDTA), D-mannitol and ethanol. In some embodiments, provided herein is a composition for use in treating a viral infection, wherein the viral infection is a nasal infection, and wherein the composition comprises a sodium salt of Ethylenediaminetetraacetic acid (EDTA), D-mannitol and ethanol. In some embodiments, provided herein is a composition for use in treating a viral infection, wherein the viral infection is a skin infection, and wherein the composition comprises a sodium salt of Ethylenediaminetetraacetic acid (EDTA), D-mannitol and ethanol.

In some embodiments, the composition for use in treating a viral infection reduces the viral titer in a subject by the percentages or log 10 values and in the time period as described above.

It is to be understood that the foregoing descriptive matter is illustrative of the disclosure and not a limitation. While considerable emphasis has been placed herein on the particular features of this disclosure, it will be appreciated that various modifications can be made, and that many changes can be made in the preferred embodiments without departing from the principles of the disclosure. Those skilled in the art will recognize that the embodiments herein can be practiced with modification within the spirit and scope of the embodiments as described herein. Similarly, additional embodiments and features of the present disclosure will be apparent to one of ordinary skill in art based upon description provided herein.

Descriptions of well-known/conventional methods/steps and techniques are omitted so as to not unnecessarily obscure the embodiments herein. Further, the disclosure herein provides for examples illustrating the above-described embodiments, and in order to illustrate the embodiments of the present disclosure certain aspects have been employed. The examples used herein for such illustration are intended merely to facilitate an understanding of ways in which the embodiments herein may be practiced and to further enable those of skill in the art to practice the embodiments herein. Accordingly, the following examples should not be construed as limiting the scope of the embodiments herein.

### EXAMPLES

### Example 1: Method of Preparing the Formulation

The desired anti-infective compositions of the present disclosure were formulated by mixing sodium salt(s) of EDTA and D-mannitol in an aqueous solvent, such as distilled water. The pH of the mixture was increased to about 7 by addition of sodium hydroxide solution. Once EDTA and D-mannitol were completely dissolved resulting in a clear solution, the required volume of ethanol was added. Then, the solution pH was adjusted to the required value. Finally, the formulation's volume was brought to the required level by addition of the aqueous solvent, such as distilled water.

In the alternative, the desired anti-infective compositions of the present disclosure are formulated by dissolving the sodium salt(s) of EDTA, Ethanol and D-Mannitol in a solvent to provide a concentrated, solubilized solution, and additional solvents or components may then be added, or the solubilized composition may be formulated in a form other than a solution, such as a topical preparation. The anti-infective composition may then be sterilized using conventional means, such as filtration and/or ultra-filtration, and other means. The compositions are preferably formulated using pharmacopoeia grade materials.

### Example 2: Antiviral Efficacy Against SARS Cov-2

The antiviral efficacy test was done in two steps:

### Virucidal Assay:

An infection media containing the SARS Cov-2 virus at about 10⁵ PFU/ml was prepared. The test solution of 90 μL was mixed with 10 μL of virus in 96 well plate and incubated for 3 minutes or 10 minutes at room temperature. Similarly, 70% ethanol (positive control) and media only (negative control) were mixed with virus and incubated for the required contact time at room temperature. The 10-fold serial dilutions of test material mix and controls after the completion of incubation were prepared. The viral load in each of the dilutions was determined by plaque assay for each of the samples. The cell only control (without virus) was maintained throughout the assay.

### Plaque Assay

The Vero E6 cells were plated onto 96-well plates and incubated overnight. After incubation, each dilution of the virus-test sample, virus-positive control, virus-negative control and cell only control prepared in virucidal assay were exposed to the Vero E6 cells for about 1 hour followed by shaking after every 15 minutes. After exposure, medium containing the test material and the virus were removed from the wells and mixture of growth media and carboxy methylcellulose was added to each of the wells and incubated for 3 days. After incubation, carboxy methylcellulose was removed from each of the wells and cells were fixed by the addition of formaldehyde. After fixation, the cells were stained using crystal violet for plaque visualization. The numbers of plaques were counted in a dilution at which clear plaques are visible and the PFU/0.1 ml was determined. The residual viral load was calculated as follows:
a) Counted the number of plaques in a well
b) Multiplied the number of plaques with the dilution factor
c) Multiplied the number obtained in step b by 2, to obtain the PFU/0.1 ml since only 50 uL of virus was utilized for experiment.
d) Converted the PFU into log 10 values to obtain the residual viral load.

The following table shows the results obtained for the antiviral efficacy against SARS-Cov-2:

TABLE 1
Data showing anti-viral activity using the present formulation

| | Residual Viral Load |
|---|---|
| Test Sample Vial | (Log PFU/0.1 ml) |
| Virus Only (Average - Negative control) | 4.02 |
| Ethanol 70% (Positive Control) | 0.00 |
| EDTA 5% | 3.40 |
| Ethanol 25% | 2.26 |
| Present formulation | 0.00 |
| 5 wt % EDTA + 25 vol/vol % Ethanol + 0.75 wt % | |
| D-Mannitol in DI water | |

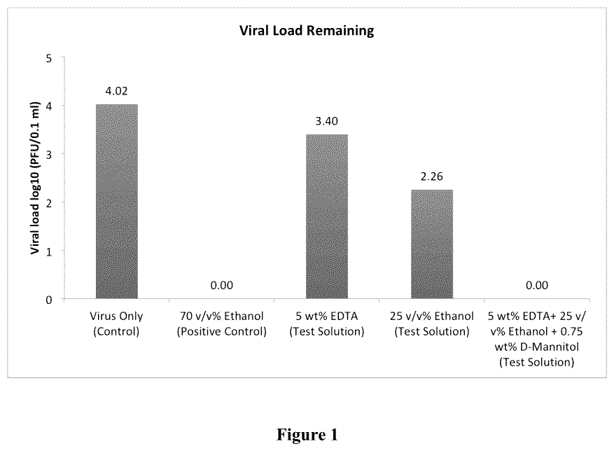

The above data shows that the viral load was completely eliminated when the sample was exposed to the formulation of the present disclosure for 3 minutes. Similar results were obtained when the virus was incubated with the formulation of the present disclosure for 10 minutes. The data listed in Table 1 and illustrated in FIG. 1 shows that the present formulation is as effective as 70% ethanol (EtOH). The present formulation demonstrates a synergy of greater than 2 log₁₀ (i.e., 2 logs higher than the most active ingredient in the formulation which is ethanol 25%). Further, the composition comprising EDTA, ethanol and D-Mannitol in the amounts described herein provide enhanced safety due to significantly lower percentage of ethanol as opposed to standard >60% ethanol used. Without being bound to theory, D-Mannitol is expected to enhance the mucociliary clearance as well as the residence time of the formulation on the surface, while EDTA salt(s) along with ethanol are expected to kill the virus thus resulting in a reduced viral load thereby reducing the spread of infection.

From the foregoing, the present disclosure may be embodied in forms other than those discussed above, and the scope of the present disclosure should be determined by the claims and the detailed discussion presented above.

The present disclosure has following advantages:
a) The present formulation has a high antiviral activity.
b) The antiviral efficacy of the present formulation is equivalent to 70% ethanol; at the same time, the amount of ethanol employed in the formulation is significantly lower.
c) The present formulation has enhanced antiviral efficacy compared to EDTA or ethanol alone.
d) The present formulation exhibits a synergistic antiviral activity when compared with EDTA or ethanol alone.

1. A method for reducing a viral titer in a subject, comprising administering to the subject a composition comprising:
a. a sodium salt of Ethylenediaminetetraacetic acid (EDTA);
b. D-mannitol; and
c. Ethanol.

2. The method of claim 1, wherein the sodium salt of EDTA is present at a concentration ranging from about 4% to about 8% (w/v).

3. The method of claim 1, wherein D-mannitol is present at a concentration ranging from about 0.5% to about 10% (w/v).

4. The method of claim 1, wherein ethanol is present at a concentration ranging from about 12.5% to about 25% (v/v).

5. The method of claim 1, wherein the composition has a pH ranging from 4 to 12.

6. The method of claim 1, wherein the sodium salt of EDTA is present at a concentration of about 5% (w/v).

7. The method of claim 1, wherein D-mannitol is present at a concentration of about 0.75% (w/v).

8. The method of claim 1, wherein ethanol is present at a concentration of about 25% (v/v).

9. The method of claim 1, wherein the composition has a pH of 7.5+/−0.5.

10. The method of claim 1, wherein the sodium salt of EDTA is present at a concentration ranging from about 4% to about 8% (w/v), D-mannitol is present at a concentration ranging from about 0.5% to about 10% (w/v), and ethanol is present at a concentration ranging from about 12.5% to about 25% (v/v).

11. The method of claim 1, wherein the sodium salt of EDTA is present at a concentration of about 5% (w/v), D-mannitol is present at a concentration of about 0.75% (w/v), and ethanol is present at a concentration of about 25% (v/v).

12. The method of claim 1, wherein the composition is administered intranasally, enterally, sublingually, intramuscularly, intravenously, intrathecally, topically, as irrigation, subcutaneously, as inhalation, or mucosally.

13. The method of claim 1, wherein the administration of the composition shows an antiviral activity against enveloped or non-enveloped viruses selected from the group consisting of coronavirus, influenza, respiratory syncytial virus (RSV), rhinovirus, or variants thereof.

14. The method of claim 13, wherein the coronavirus is SARS-CoV-2 or variants thereof.

15. The method of claim 1, wherein the administration of the composition reduces the viral titer in the subject by at least 60%.

16. The method of claim 1, wherein the administration of the composition reduces the viral titer in the subject by at least 60% in at least 10 minutes, after administering the composition.

17. The method of claim 1, wherein the viral titer is reduced by at least 1 log 10 as measured according to Plaque Assay testing method.

18. The method of claim 1, wherein the composition is administered at least once a day.

19. The method of claim 1, wherein the sodium salt of EDTA is selected from the group consisting of di-sodium salt of Ethylenediaminetetraacetic acid (EDTA), tri-sodium salt of Ethylenediaminetetraacetic acid (EDTA), tetra-sodium salt of Ethylenediaminetetraacetic acid (EDTA), and a combination thereof.
